(19) United States
(12) Patent Application Publication
(10) Pub. No.: US 2008/0227693 A1
(43) Pub. Date: Sep. 18, 2008

(54) Cyclic Peptide Isolation by Spray Drying

(75) Inventors: Rowena Fernandez Choudrie, Edison, NJ (US); Kaushik J. Dave, Edison, NJ (US); Steven R. Johnson, Monmouth Junction, NJ (US)

Correspondence Address: PALATIN TECHNOLOGIES, INC., 4-C CEDAR BROOK DRIVE, CEDAR BROOK CORPORATE CENTER, CRANBURY, NJ, 08512, US

(73) Assignee: PALATIN TECHNOLOGIES, INC., Cranbury, NJ (US)

(21) Appl. No.: 12/064,896

(22) PCT Filed: Aug. 29, 2006
(86) PCT No.: PCT/US06/33465

§ 371 Date: Feb. 26, 2008

Related U.S. Application Data
(60) Provisional application No. 60/712,276, filed on Aug. 29, 2005.

(51) Int. Cl. A61K 38/12; C07K 1/14; C07K 7/64

(52) U.S. Cl. 514/9; 530/317; 530/344

(57) ABSTRACT

Methods for isolation of a synthetic cyclic peptide by spray drying, including spray drying at elevated temperatures, products made by the methods, and synthetic cyclic peptides preparations with defined characteristics, including an essentially amorphous acid addition salt of Ac-Nle-cyclo(-Asp-His-D-Phe-Arg-Trp-Lys)-OH in the form of a fine powder with a particle diameter of about 2 to about 20 microns.

### CROSS-REFERENCE TO RELATED APPLICATION

This application claims priority to and the benefit of the filing of U.S. Provisional Patent Application Ser. No. 60/712,276, entitled “Cyclic Peptide Isolation by Spray Drying”, filed on Aug. 29, 2006, and the specification and claims thereof are incorporated herein by reference.

### BACKGROUND OF THE INVENTION

1. Field of the Invention (Technical Field)

The present invention relates to methods for isolation of cyclic peptide active pharmaceutical ingredient (“API”) by spray drying, and more particularly to methods of concentration and isolation, following synthesis and initial purification, of the cyclic peptide Ac-Nle-cyclo(-Asp-His-D-Phe-Arg-Trp-Lys)-OH, by means of spray drying.

2. Background Art

There are two main approaches to the synthetic manufacture of small peptides, and specifically cyclic peptides. One involves solution or liquid phase peptide synthesis, where amino acid residues in solution are linked by peptide bonds, with reactive groups not involved in the peptide bond formation, such as the amino group of the N-terminal residue, the carboxy group of the C-terminal residue, and similar or other reactive groups in the amino acid side chains, protected by suitable protecting groups. The other approach involves solid phase peptide synthesis, in which synthesis is carried out on an insoluble solid matrix. Protecting groups are employed for reactive side chains. The general methodology of solid phase synthesis is well known in the art. Merrifield, R. B., Solid phase synthesis (Nobel lecture). Angew Chem 24:799-810 (1985) and Barany et al., The Peptides, Analysis, Synthesis and Biology, Vol. 2, Gross, E. and Meienhofer, J., Eds. Academic Press 1-284 (1980).

Other methods are known, including various recombinant and semi-synthetic methodologies. However, for small peptides, such as heptapeptides, and in particular for heptapeptides cyclized by the formation of a lactam bridge, synthetic routes are generally the most cost effective and efficient methodologies.

In making a cyclic peptide, at some point in the synthetic process the relevant reactive groups are linked to form a cyclic peptide. It is possible to do this in solution or as part of a solid phase synthetic methodology. For example, the cyclic peptide Ac-Nle-cyclo(-Asp-His-D-Phe-Arg-Trp-Lys)-NH₂ has been reported to have been made by sequential solid phase synthetic methodology employing Boc chemistry, followed by solution phase lactamization to form the cyclic peptide. Al-Obeidi F., de L. Castrucci A. M., Hadley M. E., Hruby V. J. J. Med. Chem. 32:2555-2561 (1989); Al-Obeidi F., Hadley M. E., Pettitt B. M., Hruby V. J. J. Am. Chem. Soc. 111:3413-1316 ((1989). In other studies the same peptide was both synthesized and cyclized on solid phase, utilizing Fmoc chemistry for synthesis. Grieco P., Balse-Srinivasan P., Han G., Weinberg D., MacNeil T., Van der Ploeg, L. H. T., Hruby, V. J. J. Peptide Res. 62:199-206 (2003); Grieco P., Gitu P. M., Hruby V. J. J. Peptide Res. 57:250-256 (2001). In a recent report, both Ac-Nle-cyclo(-Asp-His-D-Phe-Arg-Trp-Lys)-NH₂ and Ac-Nle-cyclo(-Asp-His-D-Phe-Arg-Trp-Lys)-OH were made by a method involving solid phase synthesis of a partial sequence including the side chains required for lactamization, cyclizing the peptide on sold phase, completing the synthesis of the remaining groups, and deprotecting and cleaving the cyclic peptide from resin. Flora D., Mo H., Mayer J. P., Khan M. A., Yan L. Z.: Detection and control of aspartimide formation in the synthesis of cyclic peptides. Bioorganic & Medicinal Chemistry Letters 15:1065-1068 (2005). Another method of solid phase synthesis and cyclization is disclosed in International Application PCT/EP2005/010133, published on 30 Mar. 2006 as International Publication WO 2006/032457, in which allyl-type protecting groups are used on side chains intended to form a lactam bridge, with the N-terminus nitrogen protected by a base-labile protecting group. This method is exemplified for the making of Ac-Nle-cyclo(-Asp-His-D-Phe-Arg-Trp-Lys)-OH. The teachings and disclosure of each of the foregoing references are incorporated here by reference as if set forth in full.

Regardless of how the cyclic peptide is made, once made the peptide must be purified and isolated. While solid phase chemistry frequently results in coupling efficiencies in excess of 99% per cycle, nonetheless the purity of the final peptide prior to purification is rarely as high as 90% to 95%, and typically is much lower. A variety of purification methodologies are employed; most typical is reversed phase-high performance liquid chromatography (“RP-HPLC”), such as with a C₁₈ column and any of a variety of organic phases, gradients and flow rates. Other polymeric RP columns may be employed, as may other purification methodologies. For most purposes, purity in the range of 97% to 99% is desirable; for use as an API purity in excess of 98% to 99% is generally required.

Depending on the desired salt or acid form of the peptide, typically an ion exchange process is employed following purification. The result of purification and optionally ion exchange is a pure peptide acid or salt in solution. The solution may also include one or more organic solvents, but is typically an aqueous solution. Thus isolation of the cyclic peptide, a critical step in making an API, is required. Preferably the cyclic peptide API is isolated as a dry, solid powder, containing no compounds or reagents other than the cyclic peptide acid or salt itself. For peptide synthesis, the common method of isolation is lyophilization, involving subjecting the solution containing the cyclic peptide to a controlled cooling and heating cyclic under vacuum. While effective and well known in the art, this method is both time consuming, with some lyophilization cycles requiring twenty-four hours or more, and labor and equipment intensive, generally requiring placing the solution containing the cyclic peptide in suitable lyophilization containers. Large scale lyophilization equipment is bulky and expensive to both purchase and operate. It is an inherent limitation of lyophilization that it can generally only be conducted on a batch basis, and not as part of a continuous process. Thus while well known, lyophilization as a method of isolation, particularly for large volume production of cyclic peptide acids or salts, has substantial drawbacks.

An additional drawback of lyophilization is that because efficiency of lyophilization increases with higher concentrations of the cyclic peptide in solution, purification and ion exchange methodologies are frequently adapted to produce cyclic peptide at as high a concentration as possible. Excessive solution concentration, particularly methods which involve holding the cyclic peptide in local very high concentrations, may result in undesirable reactions.

Other methods of cyclic peptide API isolation are known, such as filtration or drying methods. However these methods present potential stability issues, and are frequently limited by solubility properties of the cyclic peptide. Similarly, precipitation methods to isolate cyclic peptides as a final API also have substantial limitations, including the need to precipitate solvents to at least low ppm levels.

Spray drying has been used for isolation of non-peptide organic molecules, and has been explored for use with cyclic peptides. However, on spray drying peptides and small proteins typically show loss of activity and increased aggregation. In a number of instances the peptides also partially degrade under the high temperature conditions employed for many spray drying protocols. Thus alternative methods for spray drying have been developed, such as by use of a carrier that is water soluble or water swellable and that, when anhydrous, exists as a glass with a specified glass transition temperature, as taught in U.S. Pat. No. 6,825,031 to F. Franks et al., issued Nov. 30, 2004. However, this method necessarily introduces a second material to the API, which carrier may be a carbohydrate such as glucose, maltose, maltoriose or the like, a sugar copolymer such as a copolymer of sucrose and epichlorohydrin, a synthetic polymer such as polacrylamide, or a protein or protein hydrolysate such as albumin or hydrolysis products of gelatin. Introduction of a second material to the API is not desirable in the manufacture of drugs intended for human or veterinary use.

### BRIEF SUMMARY OF THE INVENTION

In one aspect the invention provides a method for isolation of a cyclic peptide in a concentrated solution, comprising: providing an aqueous solution comprising an acid addition salt of a cyclic peptide; and spray drying the solution wherein the peptide is maintained at an air temperature of between about 45° C. to about 100° C., preferably about 60° C. to about 92° C. In one aspect the aqueous solution consists of an acid addition salt of a cyclic peptide, water and a base or acid employed for pH adjustment. In a related aspect the aqueous solution consists of an acid addition salt of a cyclic peptide and water. In another aspect the aqueous solution consists of ammonium acetate, an acetate salt of a cyclic peptide and water.

In any of the foregoing methods, in one aspect the cyclic peptide is Ac-Nle-cyclo(-Asp-His-D-Phe-Arg-Trp-Lys)-OH. Alternatively, where an acid addition salt is provided, the acid addition salt of a cyclic peptide is an acetate salt of Ac-Nle-cyclo(-Asp-His-D-Phe-Arg-Trp-Lys)-OH.

In any of the foregoing methods, in one aspect spray drying the solution at an inlet air temperature of between about 45° C. to about 100° C. comprises spray drying the solution at a temperature of over about 55° C., over about 60° C., or over about 70° C.

In any of the foregoing methods, in one aspect during spray drying the aqueous solution comprising an acid addition salt of a cyclic peptide is maintained at a temperature of between about 24° C. and 92° C., or in an alternative aspectat a temperature of between about 20° C. and 60° C.

The invention further provides a product made by any of the foregoing methods. In one aspect the product is an amorphous acid addition salt of Ac-Nle-cyclo(-Asp-His-D-Phe-Arg-Trp-Lys)-OH. In a related aspect the product is stable at ambient temperature storage or at accelerated temperature storage conditions.

In another aspect, the invention provides a method for isolation of a cyclic peptide in a concentrated solution, comprising: providing an aqueous solution consisting essentially of ammonium acetate and an acetate salt of Ac-Nle-cyclo(-Asp-His-D-Phe-Arg-Trp-Lys)-OH in water; and spray drying the solution while maintaining a spray dryer chamber air temperature of between about 45° C. to about 100° C. In this method, in another aspect spray drying the solution at an inlet air temperature of between about 45° C. to about 100° C. comprises spray drying the solution at a temperature of over about 55° C., of over about 60° C., or of over about 70° C.

In another aspect, the invention provides a composition comprising an essentially amorphous acid addition salt of Ac-Nle-cyclo(-Asp-His-D-Phe-Arg-Trp-Lys)-OH in the form of a fine powder with a diameter of about 2 to about 20 microns in diameter forming agglomerates of a diameter of about 20 to about 200 microns. In one aspect the composition is made by spray drying an aqueous solution consisting essentially of an acid addition salt of Ac-Nle-cyclo(-Asp-His-D-Phe-Arg-Trp-Lys)-OH and water. In another aspect the composition is further characterized in that composition has better flowability, less dust, less static and increased solubility compared to a composition made by lyophilization of an aqueous solution consisting essentially of an acid addition salt of Ac-Nle-cyclo(-Asp-His-D-Phe-Arg-Trp-Lys)-OH and water.

A primary object of the present invention is to provide a method for isolation of API consisting of a Ac-Nle-cyclo(-Asp-His-D-Phe-Arg-Trp-Lys)-OH.

Another object of the present invention is to provide a low cost and rapid method of isolation of API in a continuous batch mode.

Yet another object of the present invention is to provide a method of isolation of such API utilizing spray drying methodologies.

A primary advantage of the present invention is that spray drying may be employed in a composition that consists of Ac-Nle-cyclo(-Asp-His-D-Phe-Arg-Trp-Lys)-OH and water, and optionally ammonium acetate, and whether in the Ac-Nle-cyclo(-Asp-His-D-Phe-Arg-Trp-Lys)-OH is in an acetate salt form, but without employing any second materials such as carbohydrates, polymers or proteins.

Another advantage of the present invention is that it provides a formulation for spray drying a cyclic peptide wherein all components there of except the cyclic peptide, which may be a salt or acid form of cyclic peptide, evaporate or sublime upon spray drying.

Yet another advantage of the present invention is that it optionally employs ammonium acetate, which ammonium acetate evaporates or sublimes upon spray drying, leaving only a pure composition of the cyclic peptide, which may be a salt or acid form of cyclic peptide.

Other objects, advantages and novel features, and further scope of applicability of the present invention will be set forth in part in the detailed description to follow, and in part will become apparent to those skilled in the art upon examination of the following, or may be learned by practice of the invention. The objects and advantages of the invention may be realized and attained by means of the instrumentalities and combinations particularly pointed out in the appended claims.

### DETAILED DESCRIPTION OF THE INVENTION

Spray drying as an isolation method for selected cyclic peptides may be employed with cyclic peptides synthesized by any known conventional procedure for the formation of a peptide linkage between amino acids. Such conventional procedures include, for example, any solution phase procedure permitting a condensation between the free alpha amino group of an amino acid residue having its carboxyl group or other reactive groups protected and the free primary carboxyl group of another amino acid residue having its amino group or other reactive groups protected. In a preferred conventional procedure, the cyclic peptides are synthesized by solid-phase synthesis and purified according to methods known in the art. Any of a number of well-known procedures utilizing a variety of resins and reagents may be used to prepare cyclic peptides.

The process for synthesizing the cyclic peptides may be carried out by a procedure whereby each amino acid in the desired sequence is added one at a time in succession to another amino acid residue or by a procedure whereby peptide fragments with the desired amino acid sequence are first synthesized conventionally and then condensed to provide the desired peptide. The resulting peptide is then cyclized to yield a cyclic peptide of the invention. Variations on this methodology, including those disclosed in Flora D., Mo H., Mayer J. P., Khan M. A., Yan L. Z.: Detection and control of aspartimide formation in the synthesis of cyclic peptides. Bioorganic & Medicinal Chemistry Letters 15:1065-1068 (2005), incorporated here by reference, may be similarly employed.

Solid phase peptide synthesis methods are well known and practiced in the art. In such a method the synthesis of peptides of the invention can be carried out by sequentially incorporating the desired amino acid residues one at a time into the growing peptide chain according to the general principles of solid phase methods. These methods are disclosed in numerous references, including, Merrifield, R. B., Solid phase synthesis (Nobel lecture). Angew Chem 24:799-810 (1985) and Barany et al., The Peptides, Analysis, Synthesis and Biology, Vol. 2, Gross, E. and Meienhofer, J., Eds. Academic Press 1-284 (1980).

In chemical syntheses of peptides, reactive side chain groups of the various amino acid residues are protected with suitable protecting groups, which prevent a chemical side reaction from occurring at that site until the protecting group is removed. Usually also common is the protection of the alpha amino group of an amino acid residue or fragment while that entity reacts at the carboxyl group, followed by the selective removal of the alpha amino protecting group to allow a subsequent reaction to take place at that site. Specific protecting groups have been disclosed and are known in solid phase synthesis methods and solution phase synthesis methods.

Alpha amino groups may be protected by a suitable protecting group, including a urethane-type protecting group, such as benzyloxycarbonyl (Z) and substituted benzyloxycarbonyl, such as p-chlorobenzyloxycarbonyl, p-nitrobenzyloxycarbonyl, p-bromobenzyloxycarbonyl, p-biphenyl-isopropoxycarbonyl, 9-fluorenylmethoxycarbonyl (Fmoc) and p-methoxybenzyloxycarbonyl (Moz); aliphatic urethane-type protecting groups, such as t-butyloxycarbonyl (Boc), diisopropylmethoxycarbonyl, isopropoxycarbonyl, and allyloxycarbonyl. Fmoc is preferred for alpha amino protection.

Guanidino groups may be protected by a suitable protecting group, such as nitro, p-toluenesulfonyl (Tos), Z, pentamethylchromanesulfonyl (Pmc), adamantyloxycarbonyl, and Boc. Pmc is a preferred protecting group for Arg.

For solid phase synthesis, the synthesis is conventionally commenced from the C-terminal end of the peptide by coupling a protected alpha amino acid to a suitable resin. Such a starting material can be prepared by attaching an alpha amino-protected amino acid by an ester linkage to a p-benzyloxybenzyl alcohol (Wang) resin, 4-methylbenzhydryl bromide resin, 4-methoxybenzhydryl bromide resin, or a 2-chlorotrityl chloride resin, by an amide bond between an Fmoc-Linker, such as p-[(R, S)-α-[1-(9H-fluor-en-9-yl)-methoxyformamido]-2,4-dimethyloxybenzyl]-phenoxyacetic acid (Rink linker) to a benzhydrylamine (BHA) resin, or by other means well known in the art. Fmoc-Linker-BHA resin supports are commercially available and generally used when feasible. The resins are carried through repetitive cycles as necessary to add amino acids sequentially. The alpha amino Fmoc protecting groups are removed under basic conditions. Piperidine, piperazine, diethylamine, or morpholine (20-40% v/v) in N,N-dimethylformamide (DMF) may be used for this purpose.

Following removal of the alpha amino protecting group, the subsequent protected amino acids are coupled stepwise in the desired order to obtain an intermediate, protected peptide-resin. The activating reagents used for coupling of the amino acids in the solid phase synthesis of the peptides are well known in the art. After the peptide is synthesized, if desired, the orthogonally protected side chain protecting groups may be removed using methods well known in the art for further derivatization of the peptide.

Reactive groups in a peptide can be selectively modified, either during solid phase synthesis or after removal from the resin. For example, peptides can be modified to obtain N-terminus modifications, such as acetylation, while on resin, or may be removed from the resin by use of a cleaving reagent and then modified. Methods for N-terminus modification, such as acetylation, or C-terminus modification, such as amidation, are well known in the art. Similarly, methods for modifying side chains of amino acids are well known to those skilled in the art of peptide synthesis. The choice of modifications made to reactive groups present on the peptide will be determined, in part, by the characteristics that are desired in the peptide.

The peptide can, in one embodiment, be cyclized prior to cleavage from the peptide resin. For cyclization through reactive side chain moieties, the desired side chains are deprotected, and the peptide suspended in a suitable solvent and a cyclic coupling agent added. Suitable solvents include, for example DMF, dichloromethane (DCM) or 1-methyl-2-pyrrolidone (NMP). Suitable cyclic coupling reagents include, for example, 2-(1H-benzotriazol-1-yl)-1,1,3,3-tetramethyluronium tetrafluoroborate (TBTU), 2-(7-aza-1H-benzotriazol-1-yl)-1,1,3,3-tetramethyluronium tetrafluoroborate (TATU), 2-(2-oxo-1(2H)-pyridyl)-1,1,3,3-tetramethyluronium tetrafluoroborate (TBTU), Benzotriazol-1-yl-oxytripyrrolidinophosphonium hexafluorophosphate (PyBOP) or N,N′-dicyclohexylcarbodiimide/1-hydroxybenzotriazole (DCCl/HOBt). Coupling is conventionally initiated by use of a suitable base, such as N,N-diispropylethylamine (DIPEA), sym-collidine or N-methylmorpholine (NMM).

Following cleavage of cyclic peptides from solid phase following synthesis, the peptide can be purified by any number of methods, such as RP-HPLC, using a suitable column, such as a C₁₈ column. Other methods of separation or purification, such as methods based on the size or charge of the peptide, can also be employed.

Cyclic peptides employed as an API may be in the form of any pharmaceutically acceptable salt. The term “pharmaceutically acceptable salts” refers to salts prepared from pharmaceutically acceptable non-toxic bases or acids including inorganic or organic bases and inorganic or organic acids. Salts derived from inorganic bases include aluminum, ammonium, calcium, copper, ferric, ferrous, lithium, magnesium, manganic salts, manganous, potassium, sodium, zinc, and the like. Particularly preferred are the ammonium, calcium, lithium, magnesium, potassium, and sodium salts. Salts derived from pharmaceutically acceptable organic non-toxic bases include salts of primary, secondary, and tertiary amines, substituted amines including naturally occurring substituted amines, cyclic amines, and basic ion exchange resins, such as arginine, betaine, caffeine, choline, N,N′-dibenzylethylenediamine, diethylamine, 2-diethylaminoethanol, 2-dimethylaminoethanol, ethanolamine, ethylenediamine, N-ethyl-morpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine, and the like.

When the cyclic peptide is basic, acid addition salts may be prepared from pharmaceutically acceptable non-toxic acids, including inorganic and organic acids. Such acids include acetic, benzenesulfonic, benzoic, camphorsulfonic, carboxylic, citric, ethanesulfonic, formic, fumaric, gluconic, glutamic, hydrobromic, hydrochloric, isethionic, lactic, maleic, malic, mandelic, methanesulfonic, malonic, mucic, nitric, pamoic, pantothenic, phosphoric, propionic, succinic, sulfuric, tartaric, p-toluenesulfonic acid, trifluoroacetic acid, and the like. Acid addition salts of peptides are prepared in a suitable solvent from the peptide and an excess of an acid, such as hydrochloric, hydrobromic, sulfuric, phosphoric, acetic, trifluoroacetic, citric, tartaric, maleic, succinic or methanesulfonic acid. The acetate salt form is especially useful and desired for the cyclic peptide Ac-Nle-cyclo(-Asp-His-D-Phe-Arg-Trp-Lys)-OH. A conventional method for making a salt form of a peptide is by ion exchange. Ion exchange can be performed using any conventional method, such as using an ion exchange column or by means of a batch process.

Once purified and optionally converted to the desired form, the peptide can be characterized by any number of methods, such as high performance liquid chromatography (HPLC), amino acid analysis, mass spectrometry, and the like.

It has been surprisingly and unexpectedly found that select cyclic peptides, including specifically Ac-Nle-cyclo(-Asp-His-D-Phe-Arg-Trp-Lys)-OH, may be isolated by spray drying without significant damage or alteration to the peptide, and without employing any carrier or second substance, such as a glass transition carrier. Spray drying may be performed at a temperature heretofore believed to cause denaturation or other degradation of a peptide, such as spray drying at temperatures of over about 55° C., 60° C. or about 70° C. Depending on the specific spray drying device employed, the inlet gas temperature may be significantly higher, such as over about 100° C., or even over about 200° C.

In general, fluid bed and similar spray dryers maintain the temperature of the peptide within the spray dryer chamber at or near the temperature of the inlet air, and thus the inlet air temperature is the critical temperature. Other spray dryers, such as for example cyclone style spray dryers, maintain the temperature of the peptide within the spray dryer chamber, such as for example a cyclone separator, at or near the temperature of the outlet air, and thus the outlet air temperature is the critical temperature. In a cyclone style or similar spray dryer, the inlet temperature may be significantly higher without denaturation or other degradation of the peptide, since the peptide solution rapidly transits the inlet and is introduced into a spray dryer chamber, such as a cyclone separator, which is at a lower temperature.

It has further been found that this method provides the significant and desired advantage that the concentration of the peptide may be comparatively low following the purification and ion exchange steps, and thus deleterious effects which may be caused by high concentration, particularly very high local concentrations, of peptide during purification or ion exchange can readily be avoided.

While the methods disclosed herein have broader application, they have particular utility with the cyclic peptide Ac-Nle-cyclo(-Asp-His-D-Phe-Arg-Trp-Lys)-OH. This peptide is disclosed and taught in commonly owned U.S. patent application Ser. No. 10/040,547, now U.S. Pat. No. 6,794,489, entitled Compositions and Methods for Treatment of Sexual Dysfunction, filed on Jan. 4, 2002 and issued as a patent on Sep. 21, 2004, and in U.S. patent application Ser. No. 09/606,501, now U.S. Pat. No. 6,579,968, entitled Composition and Methods for Treatment of Sexual Dysfunction, which application was filed on Jun. 28, 2000 and issued as a patent on Jun. 17, 2003. Related peptides, particularly melanocortin receptor agonist peptides with utility for treatment of sexual dysfunction, which peptides may be formulated for intranasal delivery, are disclosed in commonly owned U.S. patent application Ser. No. 10/638,071, entitled Cyclic Peptide Compositions and Methods for Treatment of Sexual Dysfunction, filed on Aug. 8, 2003, and International Application No. PCT/US02/22196, International Publication No. WO 03/006620, entitled Linear and Cyclic Melanocortin Receptor-Specific Peptides, filed on Jul. 11, 2002 The teaching, specification and claims of each of the foregoing patents and patent applications is incorporated herein by reference as if set forth in full.

The spray drying method disclosed herein creates a fine powder as well as built-up agglomerates of the fine powder, thereby providing for improved flowability, reduction of static charge, reduction of dust and related fines, increased bulk density, and better solubility.

Spray drying is a process in which a homogeneous mixture of the cyclic peptide in a suitable solvent is introduced via a nozzle (e.g., a two fluid nozzle), spinning disc or rotary atomizer, an ultrasonic atomizer, or an equivalent device into a hot gas stream to atomize the solution to form fine droplets. The solvent may be an aqueous solvent, or may be a mixture of water and an organic solvent. Preferably the cyclic peptide forms a solution in the suitable solvent, which may be an aqueous mixture. Such a mixture may also contain ammonium acetate as a buffer commonly used to convert peptides into an acetate salt form. The solvent or solvents, including ammonium acetate, rapidly evaporate or sublime from the droplets, thereby initially producing a fine dry powder having particles from about 2 to about 20 microns in diameter.

The spray drying is done under conditions that result in a substantially amorphous powder of homogeneous constitution with a low moisture content. The resulting fine powder may then build up to form agglomerates, which agglomerates have a diameter of about 20 to about 200 microns.

The solutions may be sprayed dried in conventional spray drying equipment from commercial suppliers, such as Glatt Air Techniques, Büchi, Niro, Yamato Chemical Co., Okawara Kakoki Co., Fluid Air, and the like, resulting in a substantially amorphous particulate product. For the spraying process, such spraying methods as rotary atomization, pressure atomization and two-fluid atomization can be used.

The novel of the atomizer is selected such that a spray-dry composition with a suitable grain diameter is produced. Any suitable gas may be used to dry the sprayed material, such as air, nitrogen gas or an inert gas.

Any suitable air flow volume rate may be employed, such as between about 100 and 270 cfm in smaller devices. In larger scale devices, such as for commercial production of large quantities of peptide, the air flow volume may be correspondingly larger, such as between about 1000 and 2500 cfm. The temperature of the inlet of the gas used to dry the sprayed materials is such that it does not cause heat deactivation of the sprayed material; however, depending on the design and configuration of the spray drying device the inlet gas temperature may be higher than the temperature of the sprayed materials. The product temperature may be maintained between about 24° C. and about 92° C. The temperature of the outlet gas used to dry the sprayed material may vary between about 0° C. and about 120° C., preferably between about 0° C. and 60° C. However, the product temperature during spray drying is more important than the outlet gas temperature since the outlet gas temperature is, in substantial part, a consequence of the product bed temperature. The fact that inlet and product temperatures substantially above about 55° C. can be used is surprising in view of the fact that most peptides and cyclic peptides deactivate at that temperature, with nearly complete deactivation occurring typically at about 70° C.

The flow rate of the feed can similarly be varied, such as between about 10 to 20 g per minute to about 20 to 40 g per minute. The nozzle air pressure may also be varied, such as between about 1 and 3 bar. The feed itself may be any suitable sample concentration, such as between about 20 to about 100 mg per mL. The feed solution may be at any suitable pH, such as between about 3 and 5.5. The temperature of the feed solution may vary between about 20° C. to about 60° C.

Any of a variety of parameters may be examined with respect to spray dried cyclic peptide, and specifically spray dried Ac-Nle-cyclo(-Asp-His-D-Phe-Arg-Trp-Lys)-OH. This includes powder microscopy, such as looking for signs of formation of a crystalline state, measuring for increase or decrease in bulk or tap density, determining solubility, length of time to solubilize, whether the peptide stays in solution, acetate level, purity, such as by HPLC, after spray drying, X-ray diffraction, moisture content, such as by Karl Fisher analysis, and stability of the spray dried material over time and at different storage conditions.

It is particularly advantageous that the peptide, whether made by solution phase synthesis or solid phase synthesis, may be isolated without ever substantially concentrating the peptide. Thus, by way of example, the peptide, which may be Ac-Nle-cyclo(-Asp-His-D-Phe-Arg-Trp-Lys)-OH, can be synthesized and purified while leaving the concentration at each step below about 30 mg/mL, and in one aspect, below about 20 mg/mL. Peptides, in general, are highly flexible molecules which can exist in a wide spectrum of conformational or polymorphic states in solution. By leaving the concentration of the peptide dilute during synthesis and purification steps, such as for example below, preferably substantially below, saturation limits or at or below, preferably substantially below, the critical micellular concentration, the peptide is maintained in a relaxed conformational state, thus avoiding conformational and structural dynamics. The concentration limits for individual peptides may be ascertained by known empirical means, which means are known in the art. It is possible and feasible to make and purify a peptide, including a cyclic peptide such as Ac-Nle-cyclo(-Asp-His-D-Phe-Arg-Trp-Lys)-OH, while keeping the peptide in a dilute solution throughout synthesis, deprotection, cleavage, ion exchange, purification, and similar steps, such that the peptide is below the predetermined concentration limit, such as a saturation limit or critical micellular concentration limit. However, it is not practical to use lyophilization for isolation of a dilute solution, particularly in bulk or commercial scale quantities. By its nature, lyophilization is a batch process and requires a container, such as a vial, flask, tray or other container, in which the peptide solution is placed for lyophilization. Thus in one aspect the invention provides a method of isolation of a peptide in solution, wherein the peptide is maintained throughout the synthesis and purification process at or below desires concentration limits, such as at or below about 30 mg/mL or 20 mg/mL.

The invention further provides for methods where the peptide solution may be subjected to thermal treatment prior to isolation without undergoing either a freeze cycle or a concentration cycle. Thus in one aspect a peptide solution, such as a solution containing about 30 mg/mL or less of the peptide Ac-Nle-cyclo(-Asp-His-D-Phe-Arg-Trp-Lys)-OH, may be subjected to thermal treatment prior to spray drying. This thermal treatment may be at a temperature of between about 55° C. and about 70° C., and the solution may be held at this temperature for a period, such as about one hour, two hours, three hours or longer. Following thermal treatment, the peptide solution may be cooled, such as to ambient temperature or lower, and then the peptide isolated by spray drying, or alternatively the peptide solution may be utilized in a spray drying procedure without the solution undergoing cooling, or without undergoing cooling to ambient temperature.

Utilization of spray drying with a cyclic peptide, such as the peptide Ac-Nle-cyclo(-Asp-His-D-Phe-Arg-Trp-Lys)-OH, provides a number of distinct advantages. In one aspect, with spray drying there is better control of salt concentrations, such as acetate salts. In another aspect, with spray drying it is possible to conduct manufacturing on a continuous or constant throughput basis, rather than a batch process as with lyophilization methods. In yet another aspect, the dried powder resulting from spray drying has significant and substantial differences from the dried powder resulting from lyophilization, including small particle size, spherical or roughly spherical particles rather than the disk-shaped particles resulting from lyophilization, and improved parameters, such as a free flowing powder with better flowability, higher bulk density, less dust, less static and increased solubility.

### INDUSTRIAL APPLICABILITY

The invention is further illustrated by the following non-limiting examples.

### EXAMPLE 1

A Glatt GPCG 5 fluid bed spray dryer was preheated at 60° C. and 270 cfm air volume prior to spraying a 50 g/L concentrated peptide solution consisting of an acetate salt form of Ac-Nle-cyclo(-Asp-His-D-Phe-Arg-Trp-Lys)-OH in water. Spraying was initiated at 8 g/min flow rate, and gradually increased to 26 g/min. Inlet air temperature was initially at 60° C., and steadily decreased to 47° C. Atomization air was initiated at 3.0 bar, and incrementally decreased to 1.5 bar. Air volume was initiated at 270 cfm, and steadily decreased to 180 cfm. Product temperature was initially at 54° C., and steadily decreased to 31° C. The dried material was tested, and the results are tabulated below in Table 1.

TABLE 1

| | Sample 1 | Sample 2 | Sample 3 |
|---|---|---|---|
| % Purity | >99% | >99% | >99% |
| % Peptide | 88% | 88% | 87% |
| Content | | | |
| % Potency^{b} | >99% | >99% | >99% |
| % Acetic Acid | 7.2% | 7.9% | 8.1% |
| % TFA | ND | ND | ND |
| % Water | 4.8% | 5.2% | 5.3% |
| Content | | | |
| X-Ray | Amorphous^{a} | Amorphous^{a} | Amorphous^{a} |
| Diffraction | | | |
| Microscopy | 20-40 μm | 30-110 μm | 15-60 μm |

^{a}No resolved reflections indicating that the samples are amorphous.
^{b}Corrected for peptide content.
ND = None detected.

### EXAMPLE 2

A Glatt GPCG 1 fluid bed spray dryer was preheated at 60° C. prior to spraying the 50 g/L concentrated peptide solution of Example 1. Spraying was initiated at 4.5 g/min flow rate, and gradually increased to 7.5 g/min. Inlet air temperature was maintained at 60° C. Atomization air was maintained at 1.5 bar. Product temperature was initially at 51° C., and steadily decreased to maintain 25° C. The dried material was tested when dried and again after 38 days at accelerated stability conditions of 40° C. and 50° C., with no change in test results.

### EXAMPLE 3

A Glatt GPCG 5 fluid bed spray dryer was used to spray dry a 20 g/L concentration of peptide solution at pH 4.6. An acetate salt form of Ac-Nle-cylo(-Asp-His-D-Phe-Arg-Trp-Lys)-OH in water was employed. A variety of conditions were employed, as shown in Table 2 below, with the results as shown.

TABLE 2

| | Inlet Air | Spray | | | Moisture | | |
|---|---|---|---|---|---|---|---|
| Air Flow | Temp. | Rate | Atomization | Bulk density | Content | Purity | X-Ray |
| (CFM) | (° C.) | (g/min) | Air (bar) | (g/cc) | (%) | (%) | Diffraction |
| 270 | 100 | 20-40 | 3 | 0.062 | 6.7 | >99 | Amorphous |
| 270 | 100 | 10-20 | 1 | 0.052 | 7.8 | >99 | Amorphous |
| 270 | 60 | 20-40 | 1 | 0.095 | 6.9 | >99 | Amorphous |
| 270 | 60 | 10-20 | 3 | 0.091 | 6.6 | >99 | Amorphous |
| 180 | 100 | 20-40 | 1 | 0.091 | 7.3 | >99 | Amorphous |
| 180 | 100 | 10-20 | 3 | 0.071 | 5.9 | >99 | Amorphous |
| 180 | 60 | 20-40 | 3 | 0.130 | 6.7 | >99 | Amorphous |
| 180 | 60 | 10-20 | 1 | 0.094 | 6.3 | >99 | Amorphous |

### EXAMPLE 4

A Glatt GPCG 5 fluid bed spray dryer was used to spray dry a 20 g/L or 50 g/L concentration of peptide solution of Ac-Nle-cyclo(-Asp-His-D-Phe-Arg-Trp-Lys)-OH. The pH was varied in different runs from between pH 3.0 and pH 4.0 using glacial acetic acid. At pH 3.0, at either 20 g/L or 50 g/L concentration recoveries greater than 30% were observed using an air flow volume of 180 cfm, an inlet air temperature of 60° C., a spray rate initially of 10 g/min increasing to 20 g/min, and a nozzle air pressure of 1.5 bar.

Compared to the starting material, which had been isolated by lyophilization, the spray dried material exhibited equivalent purity with increased bulk and tap density. Photo microscopy revealed that the lyophilized starting material was in the form of plates, with an average particle size of 50 to 100 μm, while the spray dried material was in the form of round particles with an average particle size of less than 50 μm.

### EXAMPLE 5

A Glatt GPCG 5 fluid bed spray dryer was used to spray dry a 100 g/L concentration of peptide solution of Ac-Nle-cyclo(-Asp-His-D-Phe-Arg-Trp-Lys)-OH adjusted to pH 3.50 using glacial acetic acid. Testing was conducted using an air flow volume of 180 cfm, an inlet air temperature of 60° C., a spray rate initially of 10 g/min increasing to 20 g/min, and a nozzle air pressure of 1.5 bar. The tap density of the resulting material was 1.7 times greater than that of the untapped bulk density.

### EXAMPLE 6

A Büchi 190 spray dryer with a cyclone separator was employed which had a nozzle diameter of 0.6 mm. A 50 g/L concentration of peptide solution of Ac-Nle-cyclo(-Asp-His-D-Phe-Arg-Trp-Lys)-OH was employed, with a feed rate of 4.9 g/min and a gas flow rate of from 56 to 85 cfm with an inlet temperature of 265° C. and an outlet temperature of between 80° C. and 120° C. At temperatures between 90° C. and 120° C. dry product was obtained. With a cyclone style spray dryer, a higher inlet temperature may be employed without denaturation or other degradation of the cyclic peptide because the cyclic peptide is subject to the inlet temperature only for the period of time while transiting the inlet, with the cyclone separator temperature generally maintained at or about the outlet temperature.

The preceding examples can be repeated with similar success by substituting the generically or specifically described reactants and/or operating conditions of this invention for those used in the preceding examples.

Although the invention has been described in detail with particular reference to these preferred embodiments, other embodiments can achieve the same results. Variations and modifications of the present invention will be obvious to those skilled in the art and it is intended to cover all such modifications and equivalents. The entire disclosures of all references, applications, patents, and publications cited above and/or in the attachments, and of the corresponding application(s), are hereby incorporated by reference.

1. A method for isolation of a cyclic peptide in a solution, comprising;
providing an aqueous solution comprising an acid addition salt of a cyclic peptide; and
spray drying the solution at an inlet air temperature of over about 45° C.

2. The method of claim 1, wherein the aqueous solution consists of an acid addition salt of a cyclic peptide, water and a base or acid employed for pH adjustment.

3. The method of claim 2, wherein the aqueous solution is at a concentration of 30 mg/mL or less.

4. The method of claim 1, 2 or 3 wherein the cyclic peptide is Ac-Nle-cyclo(-Asp-His-D-Phe-Arg-Trp-Lys)-OH.

5. The method of claim 1, 2 or 3 wherein the acid addition salt of a cyclic peptide is an acetate salt of Ac-Nle-cyclo(-Asp-His-D-Phe-Arg-Trp-Lys)-OH.

6. The method of claim 1 wherein spray drying the solution at an inlet air temperature of over about 45° C. comprises spray drying the solution at a temperature of over about 55° C. but below about 100° C.

7. The method of claim 1 wherein spray drying the solution at an inlet air temperature of over about 45° C. comprises spray drying the solution at a temperature of over about 60° C. but below about 100° C.

8. The method of claim 1 wherein spray drying the solution at an inlet air temperature of over about 45° C. comprises spray drying the solution at a temperature of over about 70° C. but below about 100° C.

9. The method of claim 1 wherein during spray drying the aqueous solution comprising an acid addition salt of a cyclic peptide is maintained at a temperature of between about 24° C. and 92° C.

10. The method of claim 1 wherein during spray drying the aqueous solution comprising an acid addition salt of a cyclic peptide is maintained at a temperature of between about 20° C. and 60° C.

11. A product made by the method of claim 1.

12. The product of claim 11 which is an amorphous acid addition salt of Ac-Nle-cyclo(-Asp-His-D-Phe-Arg-Trp-Lys)-OH.

13. The product of claim 12 which is stable at ambient temperature storage or at accelerated temperature storage conditions.

14. A method for isolation of a cyclic peptide in a solution, comprising:
providing an aqueous solution consisting essentially of ammonium acetate and an acetate salt of Ac-Nle-cyclo(-Asp-His-D-Phe-Arg-Trp-Lys)-OH in water; and
spray drying the solution under conditions where the peptide is maintained in a spray dryer chamber at an air temperature of between about 45° C. to about 100° C.

15. The method of claim 14 wherein spray drying the solution at an air temperature of between about 45° C. to about 100° C. comprises spray drying the solution while maintaining a spray dryer chamber air temperature of over about 55° C. but less than about 92° C.

16. The method of claim 14 wherein spray drying the solution at an inlet air temperature of between about 45° C. to about 100° C. comprises spray drying the solution while maintaining a spray dryer chamber air temperature of over about 60° C. but less than about 92° C.

17. The method of claim 14 wherein spray drying the solution at an inlet air temperature of between about 45° C. to about 100° C. comprises spray drying the solution while maintaining a spray dryer chamber air temperature of over about 70° C. but less than about 92° C.

18. A composition comprising an essentially amorphous acid addition salt of Ac-Nle-cyclo(-Asp-His-D-Phe-Arg-Trp-Lys)-OH in the form of a fine powder with a particle diameter of about 2 to about 20 microns.

19. The composition of claim 18 wherein the fine powder forms agglomerates of a diameter of about 20 to about 200 microns.

20. The composition of claim 18 made by spray drying an aqueous solution consisting essentially of an acid addition salt of Ac-Nle-cyclo(-Asp-His-D-Phe-Arg-Trp-Lys)-OH and water.

21. The composition of claim 18 further characterized in that composition has one or more characteristics selected from the group consisting of better flowability, less dust, less static and increased solubility compared to a composition made by lyophilization of an aqueous solution consisting essentially of an acid addition salt of Ac-Nle-cyclo(-Asp-His-D-Phe-Arg-Trp-Lys)-OH and water.
